SEQUENCE LISTING (15 sequences)

SEQ ID NO: 1 - DNA, 24 bp, Artificial Sequence
  synthetic PCR amplification upstream oligonucleotide primer AampF for 5′ untranslated region (UTR)
tctccccagacgccgaggatggcc

SEQ ID NO: 2 - DNA, 24 bp, Artificial Sequence
  synthetic PCR amplification downstream oligonucleotide primer AampR for exon 4
tgtcctgggtctggtcctccccat

SEQ ID NO: 3 - DNA, 18 bp, Artificial Sequence
  synthetic PCR sequencing forward primer X2F for exon 2
tcggacccggagactgtg

SEQ ID NO: 4 - DNA, 22 bp, Artificial Sequence
  synthetic PCR sequencing reverse primer X2R for exon 2
gtttcattttcagtttaggcca

SEQ ID NO: 5 - DNA, 19 bp, Artificial Sequence
  synthetic PCR sequencing forward primer X3F for exon 3
cctctgtggggagaagcaa

SEQ ID NO: 6 - DNA, 24 bp, Artificial Sequence
  synthetic PCR sequencing reverse primer X3R for exon 3
tgttggtcccaattgtctcccctc

SEQ ID NO: 7 - DNA, 17 bp, Artificial Sequence
  synthetic PCR primer HARP1 for sequencing of HLA-A allele
ctcacaccatccagata

SEQ ID NO: 8 - DNA, 349 bp, Homo sapiens
  HLA-A allele A*01010101
atgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacgg

SEQ ID NO: 9 - DNA, 349 bp, Homo sapiens
  HLA-A allele A*03010101
gtgaaggcccagtcacagactgaccgagtggacctggggaccctgcgcggctactacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacgg

SEQ ID NO: 10 - DNA, 349 bp, Homo sapiens
  HLA-A allele A*0322
gtgaaggcccagtcacagactgaccgagtggacctggggaccctgcgcggctactacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcacccagcgcaagtgggaggcggcccatgaggcggagcagttgagagcctacctggatggcacgtgcgtggagtggctccgcagatacctggagaacgggaaggagacgctgcagcgcacgg

SEQ ID NO: 11 - DNA, 349 bp, Homo sapiens
  HLA-A allele A*290201
gtgaaggcccagtcacagactgaccgagcgaacctggggaccctgcgcggctactacaaccagagcgaggccggttctcacaccatccagatgatgtatggctgcgacgtggggtcggacgggcgcttcctccgcgggtaccggcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacgg

SEQ ID NO: 12 - DNA, 349 bp, Homo sapiens
  HLA-A allele A*2909
gtgaaggcccagtcacagactgaccgagcgaacctggggaccctgcgcggctactacaaccagagcgaggccggttctcacaccatccagatgatgtatggctgcgacgtggggtcggacgggcgcttcctccgcgggtaccggcaggacgcctacgacggcaaggattacatcgccttgaacgaggacctgcgctcttggaccgcggcggacatggcggctcagatcaccaagcgcaagtgggaggcggcccgtgtggcggagcagttgagagcctacctggagggcacgtgcgtggagtggctccgcagatacctggagaacgggaaggagacgctgcagcgcacgg

SEQ ID NO: 13 - DNA, 349 bp, Artificial Sequence
  synthetic HLA-A allele consensus sequence
gtgaaggcccagtcacagactgaccgagygracctggggaccctgcgcggctactacaaccagagcgaggccggttctcacaccatccagatratgtatggctgcgacgtggggtcggacgggcgcttcctccgcgggtaccggcaggacgcctacgacggcaaggattacatcgccytgaacgaggacctgcgctcttggaccgcggcggacatggcggctcagatcaccmagcgcaagtgggaggcggcccrtgwggcggagcagttgagagcctacctggakggcacgtgcgtggagtggctccgcagatacctggagaacgggaaggagacgctgcagcgcacgg

SEQ ID NO: 14 - DNA, 14 bp, Artificial Sequence
  synthetic HLA-A critical region (base 502)
gatcaccmagcgca

SEQ ID NO: 15 - DNA, 14 bp, Artificial Sequence
  synthetic HLA-A critical region (base 502)
atcaccaagcgcaa

(19) United States
(12) Patent Application Publication
(10) Pub. No.: US 2011/0002948 A1
(43) Pub. Date: Jan. 6, 2011

(54) IDENTIFICATION OF A NUCLEIC ACID MOLECULE

(75) Inventors: David Sayer, East Fremantle (AU); Damian Goodridge, Scarborough (AU); Steve Hodges, Cloverdale (AU); Malcolm McGinnis, Menlo Park, CA (US); Jason Stein, Piedmont, CA (US); Peter Krausa, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: CONEXIO 4 PTY LTD, East Fremantle (AU); ATRIA GENETICS INC., San Francisco, CA (US)

(21) Appl. No.: 12/303,925

(22) PCT Filed: Jun. 8, 2007
(86) PCT No.: PCT/AU2007/000805

§ 371 Date: Sep. 23, 2010

(30) Foreign Application Priority Data
Jun. 9, 2006 (AU) 2006903136
Aug. 28, 2006 (AU) 2006904661

(51) Int. Cl. A61K 39/00; C12Q 1/68; C07H 21/04

(52) U.S. Cl. 424/184.1; 435/6; 536/23.1

(57) ABSTRACT

This invention relates to the identification of a nucleic acid molecule. In particular, this invention relates to a method of using an oligonucleotide designed to a variable region of a nucleic acid molecule to identify A nucleic acid molecule.

### FIELD

This invention relates to the identification of a nucleic acid molecule. In particular, this invention relates to a method of using an oligonucleotide designed to a variable region of a nucleic acid molecule to identify the nucleic acid molecule.

### BACKGROUND

There are numerous examples of situations where it is necessary to distinguish a nucleic acid molecule from other nucleic acid molecules. For example, many genes have two or more alleles and there are numerous examples of situations where it can be necessary to identify the allele(s) of a subject. Moreover, many organisms have closely related genes or genes with similar sequence(s) yet it can be desirable to identify an organism based on these sequence(s). Furthermore it can be desirable to identify a particular nucleic acid molecule in a sample such as the product of a PCR reaction.

Taking the example of identifying an allele, there are at least 1200 HLA alleles currently known. While the degree of polymorphism within the HLA system is advantageous to the human species as a whole, there may be some drawbacks for individuals. For example, transplantation of organs and stem cells between individuals is the only known treatment for many diseases. However a consequence of the number of HLA alleles is that it is rare to find two individuals with the same HLA type. If tissue from a donor with a different HLA type is implanted into a recipient, the recipient's immune response will recognize the different HLA type in the donated tissue and the subsequent immune response may result in rejection of the donated tissue.

It is common for solid organ transplantation to occur with little or no HLA matching between a donor and recipient as rejection can be prevented or slowed down by drugs that suppress the immune system. However organ and recipient survival is shown to be increased by increasing the match in the HLA type of the donor and the recipient.

The situation is different for stem cell transplants, particularly bone marrow transplants, because precise matching of the HLA type of a donor and a recipient is required. In this case failure to precisely match the HLA type may result in the transplanted stem cells seeing the recipient as foreign and causing severe disease or even death.

Recent studies have demonstrated the importance of HLA typing in a number of other areas. For example, patients infected with HIV who use the drug Abacavir are almost certain to develop a potentially severe reaction if the patient's HLA type is HLA-B*5701. Similarly, patients who take Allopurinol will develop a severe reaction if they have HLA-B*5801.

HLA typing is also important in vaccine trials. In order for vaccines to work successfully the critical peptides of the vaccine are presented to the immune system by HLA. This requires that an individual has an HLA molecule capable of presenting the peptide to the immune system. This is defined by the amino acid sequence of the HLA molecule and the amino acid sequence determines the HLA type. If the individual's HLA type does not allow presentation of the vaccine because the vaccine has a protein fragment with a sequence that the HLA molecules of a subject cannot recognize, then the vaccine will not work in this individual.

Moreover, many diseases have been linked to certain HLA types, indicating that HLA alleles and/or genes that are linked to the HLA genes contribute to susceptibility to disease. Consequently many studies are performed that include HLA typing of patients and controls in an attempt to identify the disease susceptibility alleles and also to identify markers that can be used in the diagnosis of a disease associated with a particular allele. For example, almost all patients with Ankylosing Spondylitis (AS) have certain subtypes of HLA-B*27. Therefore excluding the presence of these subtypes virtually excludes a diagnosis of AS.

In many instances precise HLA typing is required. However when the HLA alleles of an individual are sequenced simultaneously the sequence that is obtained may be identical to two or more combinations of alleles. New alleles are constantly being described resulting in an increasing number of possible allele combinations. New strategies are required to identify nucleic acid molecules such as HLA alleles.

### SUMMARY

The invention provides a method of using an oligonucleotide to identify a nucleic acid molecule. The identification of a nucleic acid molecule may have many and varied uses, for example, the identification of an HLA allele, identification of a pathogenic microorganism, or identification of each component within a PCR product. Accordingly, in a first aspect the invention provides a method of designing an oligonucleotide for use in identifying a nucleic acid molecule, comprising the steps of:

a) identifying first and second variable regions in each of at least two nucleic acid molecules, wherein the first and/or second variable region of each nucleic acid molecule is descriptive of that nucleic acid molecule; and
b) designing an oligonucleotide which binds to the first variable region of one nucleic acid molecule and generates information of the second variable region of that nucleic acid molecule.

In some embodiments, the method comprises an initial step of aligning the at least two nucleic acid sequences before step a).

In a second aspect the invention provides a method of identifying a nucleic acid molecule, comprising the steps of:

a) combining an oligonucleotide which binds to a first variable region of the nucleic acid molecule and generates information of a second variable region of the nucleic acid molecule, wherein the first and/or second variable region of the nucleic acid molecule is descriptive of that nucleic acid molecule;
b) generating information about the nucleic acid molecule; and
c) analysing the generated information to identify the nucleic acid molecule.

In a third aspect the invention provides a method of HLA typing a subject, comprising the steps of:

a) combining a sample from the subject and an oligonucleotide which binds to a first variable region of an HLA allele of the subject and generates information of a second variable region of the allele, wherein the first and/or second variable region of the allele is descriptive of the allele;
b) generating the information about the allele; and
c) analysing the generated information to identify the allele,
wherein identifying the allele provides the HLA type of the subject.

In a fourth aspect the invention provides a method of treating a disease or disorder of a subject, comprising the steps of:

a) combining a sample from the subject and an oligonucleotide which binds to a first variable region of a nucleic acid molecule and generates information of a second variable region of the nucleic acid molecule, wherein the first and/or second variable region of the nucleic acid molecule is descriptive of the nucleic acid molecule;
b) generating the information about the nucleic acid molecule; and
c) analysing the generated information to identify the nucleic acid molecule,
wherein identifying the nucleic acid molecule indicates how to treat the disease or disorder.

In a fifth aspect the invention provides a method of diagnosis of a disease or disorder of a subject, comprising the steps of:

a) combining a sample from the subject and an oligonucleotide which binds to a first variable region of a nucleic acid molecule and generates information of a second variable region of the nucleic acid molecule, wherein the first and/or second variable region of each nucleic acid molecule is descriptive of the nucleic acid molecule;
b) generating the information about the nucleic acid molecule; and
c) analysing the generated information to identify the nucleic acid molecule,
wherein identifying the nucleic acid molecule provides a diagnosis of the disease or disorder.

In some embodiments the nucleic acid molecule is an allele.

In some embodiments information about more than two variable regions is generated.

In some embodiments of the second to fifth aspects the analysis may be performed by a computer program. The computer program may be Assign-SBT™.

In some embodiments of the second to fifth aspects 1 oligonucleotide is used. In another embodiment 4 oligonucleotides are used.

In some embodiments of the second to fifth aspects steps a) and b) may occur in the same container.

In some embodiments the first and second variable regions are separated by 1 to 1,500 nucleotides. In other embodiments the first and second variable regions are separated by 30 to 1000 nucleotides. In still other embodiments the first and second variable regions are separated by 100 to 500 nucleotides.

In some embodiments the alleles are amplified prior to step a).

The gene may be an HLA gene. In some embodiments the gene is HLA-DRB1.

### BRIEF DESCRIPTION OF THE FIGURES

FIG. 1 shows a diagram of variable regions of nucleic acid molecules where each variable region has a different sequence represented by the letters A, B, C, D and E

FIG. 2 shows a diagram of variable regions of nucleic acid molecules where each variable region has a different sequence represented by the letters A, B, C, and D

FIG. 3 shows the sequence of 4 HLA-A alleles, A*03010101, A*0322, A*290201 and A*2909, from position 270 to 620 compared to the sequence of allele A*01010101. Nucleotides that are identical to A*01010101 are represented as a dash (-) and differences are indicated. The letters A,C,G,T represent the nucleotides, Adenine, Cytosine, Guanine, and Thymidine according to standard nomenclature. The letter W indicates bases A+T, Y is C+T, R is A+G, K is G+T, M is A+C

FIG. 4 shows the binding sites of oligonucleotides (indicated by HARP1) used to identify HLA alleles A*290201, and A*2909. The small box at position 502 shows the nucleotide that is different between A*290201 and A*2909. Using the information that this HARP only sequences the A*290201 and A*2909 alleles and the sequence at position 502 the precise allele (A*290201 or A*2909) can be identified

FIG. 5 shows the critical region (base 502) within a sequence from experimental data where the combined sequence resulted in the HLA type being either A*030101+290201 or A*0322+2909.

### DETAILED DESCRIPTION OF PREFERRED EMBODIMENTS

Before describing the invention in detail, it is to be understood that it is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional molecular biology and pharmacology within the skill of the art. Such techniques are well known to the skilled worker, and are explained fully in the literature. See, eg., “Molecular Cloning: A Laboratory Manual”, 2^{nd} Ed., (ed. by Sambrook, Fritsch and Maniatis) (Cold Spring Harbor Laboratory Press: 1989); “Nucleic Acid Hybridization”, (Hames & Higgins eds. 1984); “Oligonucleotide Synthesis” (Gait ed., 1984); Remington's Pharmaceutical Sciences, 17^{th} Edition, Mack Publishing Company, Easton, Pa., USA.; “The Merck Index”, 12^{th} Edition (1996), Therapeutic Category and Biological Activity Index; and “Transcription & Translation”, (Hames & Higgins eds. 1984).

It must be noted that as used herein and in the appended claims, the singular forms “a,” “an,” and “the” include plural reference unless the context clearly dictates otherwise. Thus, for example, a reference to “an oligonucleotide” includes a plurality of oligonucleotides, and a reference to “an allele” is a reference to one or more alleles, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

Throughout the specification, the word “comprise” and variations of the word, such as “comprising” and “comprises”, means “including but not limited to” and is not intended to exclude other additives, components, integers or steps. By “consisting of” is meant including, and limited to, whatever follows the phrase “consisting of”. Thus, the phrase “consisting of” indicates that the listed elements are required or mandatory; and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

In one aspect, the invention provides a method of designing an oligonucleotide suitable for use in identifying a nucleic acid molecule. The inventors have surprisingly found that an oligonucleotide which binds to a variable region of a nucleic acid molecule and can be used to generate information about another variable region of the nucleic acid molecule can be used to identify the nucleic acid molecule.

Without wishing to be bound by any particular theory or hypothesis, the inventors have found that the generation of information about two or more variable regions of a nucleic acid molecule using a single oligonucleotide enables the identification of a substantially greater number of nucleic acid molecules compared with using an oligonucleotide which generates information about only one variable region.

A “variable region” of a nucleic acid molecule consists of sequence which differentiates the nucleic acid molecule from another nucleic acid molecule. The sequence may differ by one or more nucleotides, for example, as a result of the addition, deletion, or duplication of one or more nucleotides. Alternatively or in addition, the sequence may differ as a result of rearrangement of motifs within the nucleic acid molecule. The rearrangements may be inversions (reversal of order) or transpositions (movement of nucleotide sequences into new positions). Preferably the variable region does not merely distinguish an allele of one HLA group from alleles of another group. A group of alleles are those alleles which historically have specific combinations of sequence, also known as motifs, in common. An oligonucleotide which binds to this motif will bind to most, if not all, members of the group.

The variable region of a nucleic acid molecule may be determined by aligning the sequence of least two known nucleic acid molecules and identifying one or more regions with a different sequence to that present in one or more of the other nucleic acid molecules in the alignment. In some embodiments more than two known nucleic acid molecules are aligned.

In one embodiment the nucleic acid molecule is an allele of a gene. In another embodiment all of the known alleles of the gene are aligned.

As used herein the term “align” means that the sequences of the nucleic acid molecules are lined up, typically one below another and with introduced gaps if necessary, so that the variable region(s) are emphasized. Alignment for the purposes of the invention can be achieved in various ways that are within the skill in the art, for instance, using publicly available computer software such as CLUSTALW, BLAST, BLAST-2, ALIGN, ALIGN-2 or Megalign (DNASTAR) software. Those skilled in the art can determine appropriate parameters for measuring alignment, including any algorithms needed to achieve maximal alignment over the full length of the sequences being compared.

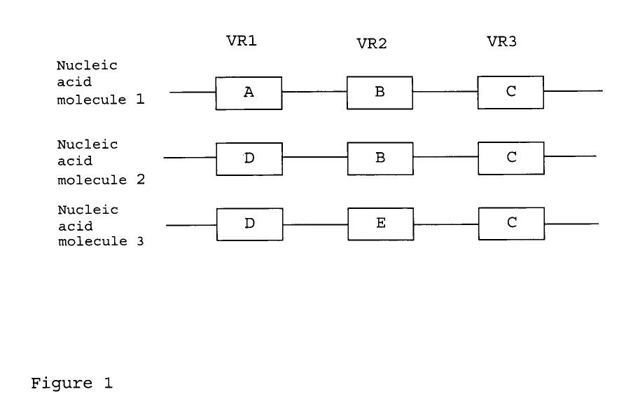

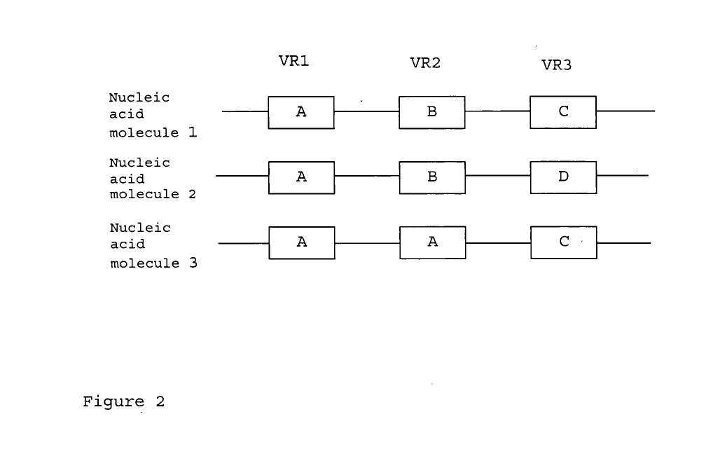

Following identification of variable regions of a nucleic acid molecule, those that are descriptive of the nucleic acid molecule are selected. The term “descriptive” means that the sequence of the variable regions can be used to distinguish one nucleic acid molecule from another nucleic acid molecule. That is, the sequence of either one of the variable regions must be unique to a particular nucleic acid molecule or the combination of variable region sequences must be unique to a particular nucleic acid molecule, thereby allowing the nucleic acid molecules to be distinguished. Examples of these scenarios are provided in FIGS. 1 and 2 respectively.

As shown in FIG. 1, nucleic acid molecule 1 can be distinguished from nucleic acid molecules 2 and 3 because it has a unique “A” sequence in variable region 1 (VR1). Similarly nucleic acid molecule 3 can be distinguished from nucleic acid molecules 1 and 2 because it has a unique “E” sequence in variable region 2 (VR2). Nucleic acid molecule 2 can be distinguished from nucleic acid molecules 1 and 3 because it has neither the “A” or “E” sequence in variable region 1 or 2.

As shown in FIG. 2, nucleic acid molecule 1 can be distinguished from nucleic acid molecule 2 because it has a “C” sequence in variable region 3 (VR3) and can be distinguished from nucleic acid molecule 3 because it has a “B” sequence in variable region 2. Nucleic acid molecule 2 can be distinguished from nucleic acid molecules 1 and 3 because it has a “B” sequence in variable region 2 and a “D” sequence in variable region 3. Nucleic acid molecule 3 can be distinguished from nucleic acid molecules 1 and 2 because it has an “A” sequence in variable region 2 and a “C” sequence in variable region 3.

This is in contrast to a situation where a conserved region of a nucleic acid molecule is identified and selected, optionally in combination with a variable region of the nucleic acid molecule. A conserved region is one which consists of sequence which is common to the majority, if not all, of the nucleic acid molecules in an alignment. In this case a particular nucleic acid molecule can only be identified if the oligonucleotide generates information about a variable region which is unique to the nucleic acid molecule.

In some embodiments the first and second variable regions of the nucleic acid molecule are separated by 1 to 1,500 nucleotides, as this is the amount of sequence information which can typically be generated by one sequencing primer. In other embodiments the first and second variable regions are separated by 30 to 1000 nucleotides. In other embodiments the first and second variable regions are separated by 100 to 500 nucleotides.

The sequence of the variable region is used to design an oligonucleotide which can identify the nucleic acid molecule comprising that variable region. As used herein, an “oligonucleotide”, “oligonucleotide primer”, or “oligonucleotide probe” is a short-length (typically between 2 and 100 nucleotides), single- or double-stranded polydeoxynucleotide that is chemically synthesised by known methods (involving, for example, triester, phosphoramidite, or phosphonate chemistry), such as described by Engels, et al., Agnew. Chem. Int. Ed. Engl. 28:716-734 (1989). Typically they are then purified, for example, by polyacrylamide gel electrophoresis.

An oligonucleotide identified by the method of the invention is designed so that it can be used to identify a nucleic acid molecule. In some embodiments the oligonucleotide will be used as a sequencing primer and/or an amplification primer. In some embodiments the oligonucleotide is both an amplification primer and a sequencing primer.

An amplification primer is an oligonucleotide that has a complementary sequence to a given DNA sequence and that is used to initiate replication by DNA polymerase. A sequencing primer is an oligonucleotide that can be extended in an enzymatic reaction to produce DNA fragment that is complimentary to the DNA fragment to which the oligonucleotide binds.

In some embodiments the amplification and/or sequencing reaction(s) occur in the one container. As used herein “designed” means that a suitable sequence is determined. Therefore designing an oligonucleotide for use in the invention means that the sequence of the oligonucleotide is determined. The sequence is determined so that the oligonucleotide binds to a first variable region of a nucleic acid molecule and generates information about the second variable region of the nucleic acid molecule. The oligonucleotide may have a degenerate sequence.

Methods of designing oligonucleotides are known to the person skilled in the art and will depend in part on the intended use of the oligonucleotide. For example, the length of an oligonucleotide intended for use as a sequencing primer is typically between 16 and 150 nucleotides, with the optimal being 16-25 nucleotides, have a G-C content of 40-60%, a melting temperature of between 55° C. and 75° C., and should not display undesirable self-hybridisation. In some embodiments the oligonucleotide has 12 nucleotides.

Alternatively or in addition the oligonucleotide may be designed for use as an amplification primer. The person skilled in the art will appreciate that similar criteria should be applied to the design of oligonucleotides for amplifying DNA. For example, important criteria to consider include primer length, melting temperature, specificity, complementary primer sequences, G-C content and polypyrimidine (T, C) or polypurine (A, G) stretches, and 3′ sequence.

Numerous computer programs are available which are suitable for designing oligonucleotides for a particular purpose. For example, “Oligo” (National Biosciences, Inc, Plymouth Minn., USA), MacVector (Kodak/IBI), and the GCG suite of sequence analysis programs may be used.

The designed oligonucleotide will be used to identify a nucleic acid molecule. In some embodiments more than one nucleic acid molecule can be identified substantially simultaneously, by including the nucleic acid molecules and the required primers in the one container.

The term “nucleic acid molecule” includes both DNA and RNA molecules and DNA/RNA hybrid molecules. As used herein a “DNA” molecule includes any type of DNA, such as genomic DNA or cDNA. Similarly, “RNA” may be any class of RNA, including messenger RNA (mRNA), transfer RNA (tRNA), or ribosomal RNA (rRNA).

A “double-stranded DNA or RNA molecule” refers to the polymeric form of deoxyribonucleotides (adenine, guanine, thymine, cytosine, or uridine) in a double-stranded helix. This term refers only to the primary and secondary structure of the molecule, and does not limit it to any particular tertiary structure. Thus, this term includes double-stranded DNA and RNA found inter alia in linear DNA or RNA molecules (eg restriction fragments), viruses, plasmids, and chromosomes. In discussing the structure of particular double-stranded DNA or RNA molecules, sequences may be described herein according to the normal convention of giving only the sequence in the 5′ to 3′ direction along the non-transcribed strand of DNA or RNA, eg the strand having a sequence homologous to the mRNA.

The DNA or RNA may be present in a sample from a subject. The term “sample” as used herein includes any sample containing DNA and/or RNA molecules. For example, a sample may be biological material of a subject as all biological material contains genes and nucleic acid molecules. Preferably, the biological material is cells, tissue, or fluid isolated from bone marrow, plasma, serum, spinal fluid, lymph fluid, the external sections of the skin, respiratory, intestinal, and genitourinary tracts, tears, saliva, milk, whole blood, blood cells, tumours, organs, and also includes samples of in vivo cell culture constituents, including but not limited to conditioned medium resulting from the growth of cells in cell culture medium, putatively virally infected cells, recombinant cells, and cell components. In some embodiments the biological material is blood. The DNA or RNA may be present in a sample containing microorganisms.

The term may also include genes and nucleic acid molecules which have been isolated or purified from at least one other component of the sample and may also include amplified DNA.

An “allele” is any of two or more alternative forms of a gene that occupy the same locus on a chromosome. Each subject has two alleles for each gene. These may be the same or different from each other.

As used herein a “gene” is a length of DNA which encodes a particular protein or RNA molecule. The term “nucleic acid molecule” means a DNA or RNA molecule. RNA sequence corresponds to DNA sequence and therefore either DNA or RNA can be used to identify an allele. Nucleic acid molecule and gene sequences disclosed herein may or may not include the 5′ and 3′ untranslated regions of the gene.

In some embodiments, such as when only low levels of DNA are present in the sample, nucleic acid molecules in the sample may be amplified, such as by PCR, prior to combining the molecules and an oligonucleotide identified by a method of the invention. “Polymerase chain reaction,” or “PCR,” as used herein generally refers to a method for amplification of a desired nucleotide sequence in vitro. In general, the PCR method involves repeated cycles of primer extension synthesis in the presence of PCR reagents, using two PCR primers capable of hybridizing preferentially to a template DNA. Typically, the PCR primers used in the PCR method will be complementary to nucleotide sequences within the template at both ends of or flanking the nucleotide sequence to be amplified, although PCR primers complementary to the DNA sequence to be amplified also may be used. See Wang, et al., in PCR Protocols, pp. 70-75 (Academic Press, 1990); Ochman, et al., in PCR Protocols, pp. 219-227; Triglia, et al., Nucl. Acids Res. 16:8186 (1988).

One nucleic acid molecule suitable for use in the invention is a gene from the HLA system. The HLA (human leukocyte antigen) genes encode proteins which are part of the HLA system and initiate an immune response in a subject by presenting the fragments of foreign, or host in the case of autoimmune response, proteins to the immune system. For example, following viral infection of a cell, fragments of virus proteins are loaded into a binding groove in the HLA molecule within the cell and the HLA then travels to the cell surface where it sits until an immune cell recognises that the HLA molecule is presenting a foreign protein. The immune system then destroys the cell that has been infected by the virus to prevent the infection of more cells and subsequent death of the individual. The HLA:protein-fragment interaction is specific so that only proteins or peptides of a specific amino acid sequence are loaded into a certain HLA molecule. The number of possible foreign protein or peptide sequences is almost infinite and therefore the HLA system has evolved to a great level of diversity in order to meet any immunological challenge. This diversity exists at an individual level and at a species level. At an individual level there are several different HLA genes. At a species level these genes have different types between different individuals. Typical HLA genes are HLA-A, HLA-B and HLA-C. These are known as the class I genes. HLA-DRB1, HLA-DQB1, HLA-DPB1 are typical HLA class II genes. Other class II genes include HLA-DRB3, HLA-DRB4, HLA-DRB5, HLA-DQA1, HLA-DRA1 and HLA-DPA1. HLA-B is the most diverse HLA allele and currently there are 809 HLA-B alleles. The invention may be used to identify alleles of any HLA gene as all HLA genes have alleles comprising variable regions.

In some embodiments a HLA-DRB1 allele of a subject is identified. HLA-DR proteins (major histocompatibility complex, class II,) belongs to the HLA class II beta chain paralogues. It is a heterodimer consisting of an alpha (DRA) and a beta chain (DRB), both anchored in the membrane. It plays a central role in the immune system by presenting peptides derived from extracellular proteins. It is expressed in antigen presenting cells (APC: B lymphocytes, dendritic cells, macrophages). The beta chain is approximately 26-28 kDa. It is encoded by 6 exons, exon one encodes the leader peptide, exons 2 and 3 encode the two extracellular domains, exon 4 encodes the transmembrane domain and exon 5 encodes the cytoplasmic tail. Within the DR molecule the beta chain contains all the polymorphisms specifying the peptide binding specificities. Hundreds of DRB1 alleles have been described and typing for these polymorphisms is routinely done for bone marrow and kidney transplantation. DRB1 is expressed at a level five times higher than its paralogues DRB3, DRB4 and DRB5. DRB1 is present in all individuals. Allelic variants of DRB1 are linked with either none or one of the genes DRB3, DRB4 and DRB5. There are 5 related pseudogenes: DRB2, DRB6, DRB7, DRB8 and DRB9.

Numerous other nucleic acid molecules are also suitable for identification using the invention. For example, nucleic acid molecules from Human Immunodeficiency Virus or Hepatitis C could be identified. Additional human genes include ABO red blood cell blood groups, disease genes responsible for such diseases as Cystic Fibrosis and cancer and other highly polymorphic gene families such as antibody or T-cell receptor genes

Once the oligonucleotide has been designed based on the variable region of a nucleic acid molecule, it will bind to the variable region of the nucleic acid molecule to which it was designed. In some embodiments the binding is specific binding using stringent hybridisation conditions. However, in some embodiments other hybridisation conditions may be used, such as when the nucleic acid molecule to be identified is not closely related to other nucleic acid molecules in the sample.

Defining appropriate hybridisation conditions is within the skill of the art. See eg., Maniatis et al., DNA Cloning, vols. I and II. Nucleic Acid Hybridisation. However, briefly, “stringent conditions” for hybridisation or annealing of nucleic acid molecules are those that (1) employ low ionic strength and high temperature for washing, for example, 0.015M NaCl/0.0015M sodium citrate/0.1% sodium dodecyl sulfate (SDS) at 50° C., or (2) employ during hybridisation a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75M NaCl, 0.075M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC and 0.1% SDS.

Regardless of whether the oligonucleotide generates information about a first and second variable region of the nucleic acid molecule, provided the oligonucleotide binds to the nucleic acid molecule information will be generated about the nucleic acid molecule. That is, it can be determined whether the sequence to which the oligonucleotide was designed is present or absent in the nucleic acid molecule, which generates information about the identity of the nucleic acid molecule.

In some embodiments the hybridization and subsequent generation of information occurs in the one container. That is, the sample and all of the oligonucleotides required to identify the nucleic acid molecule are present in the container in which hybridisation occurs.

As used herein the term “generates information” means that the oligonucleotide which binds to the first variable region allows identification of the second variable region. Therefore, the oligonucleotide which binds to the first variable region can be used to generate information about the second variable region. In some embodiments the generated information is sequence information and the oligonucleotide which binds to the first variable region can be use as a sequencing primer to generate sequence information about the second variable region.

Any sequencing reaction can be used to sequence the information generated by the oligonucleotide. Examples of sequencing reactions include those based on classic techniques such as Sanger et al., 1977. Any of a variety of automated sequencing procedures can also be used (Naeve et al., 1995) including sequencing by mass spectrometry (Cohen et al., 1996; Griffin and Griffin, 1993; Koster, WO94/16101, 1994).

The generated information may be analysed to identify the nucleic acid molecule. As used herein “analysed” means that the generated information is correlated with the corresponding variable regions.

The analysis may be a manual analysis or performed by a computer program, particularly where the number of nucleic acid molecules to be distinguished is large. In some embodiments the computer program is the Assign-SBT™ program (Conexio Genomics).

The generated information will be used to identify the nucleic acid molecule. This may in turn have other uses, such as in tissue typing and the prognosis, diagnosis, prevention, and/or treatment of a disease or identification of genetic material for other reasons such as epidemiology, evolution and population migration studies or forensics.

As used herein a “subject” means any subject, as nucleic acid molecules are known to be associated with diseases in many subjects. Moreover, HLA and most other human genes have homologues that are known in many subjects, for example in zebra fish, monkeys, chimpanzees, gorillas, and dogs.

The subject may be a human or a mammal of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), horses, and birds including those kinds of birds that are endangered, kept in zoos, and fowl, and more particularly domesticated fowl, eg., poultry, such as turkeys, chickens, ducks, geese, guinea fowl, and the like, as they are also of economical importance to humans. The term does not denote a particular age. Thus, both adult and newborn and pre-natal subjects are intended to be covered.

Identification of the allele(s) of a subject may be used in HLA typing. “HLA typing” is a test which determines the compatibility of tissue of two subjects, typically a donor and a recipient. For example, the HLA type of donor and recipient will typically be determined prior to tissue transplantation.

Identification of a nucleic acid molecule may be used in the diagnosis, treatment, prognosis, and/or prevention of a disease, because many diseases and disorders are associated with a particular nucleic acid molecule. For example, the HbS allele of the beta globin gene is known to cause sickle cell disease, the HD allele is known to cause Huntington's disease, and multiple CFTR alleles are known to cause Cystic Fibrosis. Moreover the identification of a nucleic acid molecule from a pathogenic microorganism can be used in the diagnosis, treatment, prognosis, and/or prevention of a disease caused by a microorganism.

“Disease” as used herein is a general term used to refer to any departure from health in which a subject suffers. A “disorder” refers to an abnormal functioning of a function or part of the body of a subject.

The disease may be any disease associated with a particular nucleic acid molecule, including AIDS, hepatitis, Ankylosing Spondylitis (AS), sickle cell disease, Huntington's disease, and Cystic Fibrosis. A medical or veterinary practitioner, after being provided with the identification of the nucleic acid molecule may prescribe appropriate treatment for the subject according to his or her knowledge and skill.

The term “diagnosis” means the process of identifying a disease or disorder by its symptoms, via laboratory tests (including genotypic tests) or through physical findings. The identification of a nucleic acid molecule can be used in the diagnosis of a disease associated with the nucleic acid molecule.

As used herein “treatment” or “treating” means any treatment of a disorder or disease in a subject by administering a medicament to the subject following the identification of a nucleic acid molecule, including the use of pharmacogenomics. “Treatment” and “treating” includes: (a) inhibiting the disorder or disease, i.e., arresting its development; or (b) relieving or ameliorating the symptoms of the disorder or disease, i.e., cause regression of the symptoms of the disorder or disease. The effect may be therapeutic in terms of a partial or complete cure of the disorder or disease.

The term “prognosis” shall be taken to mean an indicator of the likelihood of progression of a disease or disorder diagnosed in a subject or the likelihood of a subject developing the disease or disorder. For example, depending upon the nucleic acid molecule identified by a method of the invention, a subject might be identified as likely to develop a particular disease or disorder.

The invention will now be further described by way of reference only to the following non-limiting examples. It should be understood, however, that the examples following are illustrative only, and should not be taken in any way as a restriction on the generality of the invention described above.

### Example 1

### Method for Identifying a HLA Allele

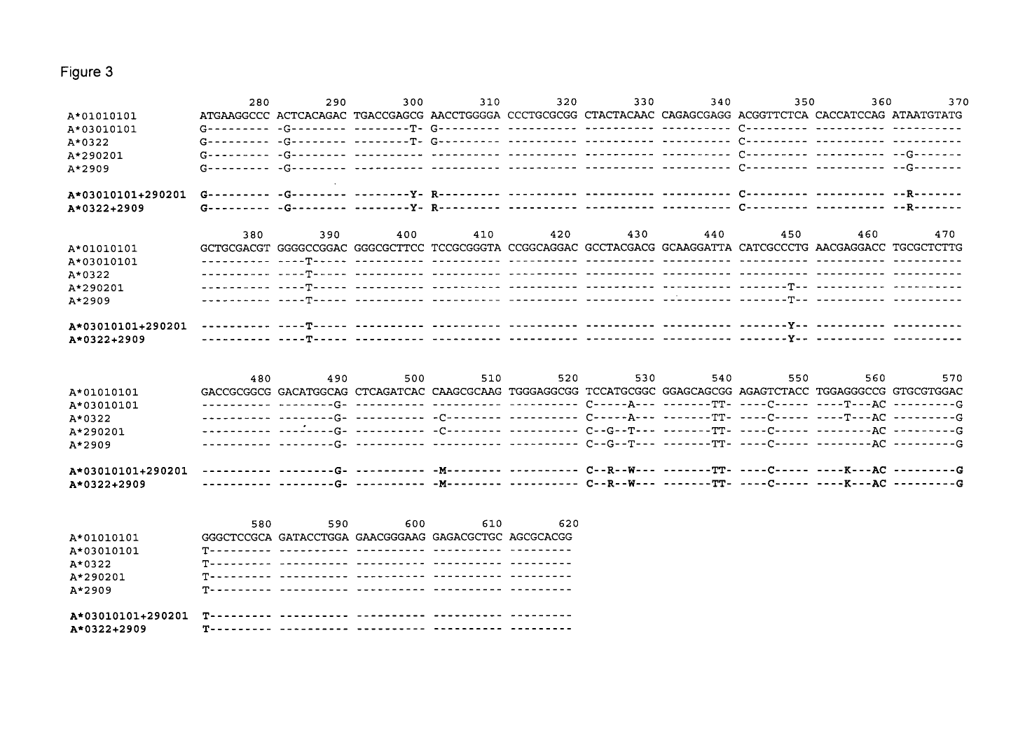

Variable regions of HLA alleles were identified by aligning alleles as shown in FIG. 3. Each allele has a unique sequence. However the combined sequence of A*03010101+290201 is identical to the combined sequence of A*0322+2909. Thus if this combined sequence was obtained when sequencing the HLA type of an individual, either A*01010101+290201 or A*0322+2909 may be present. This result may be of insufficient resolution or excessively ambiguous for some applications of HLA typing.

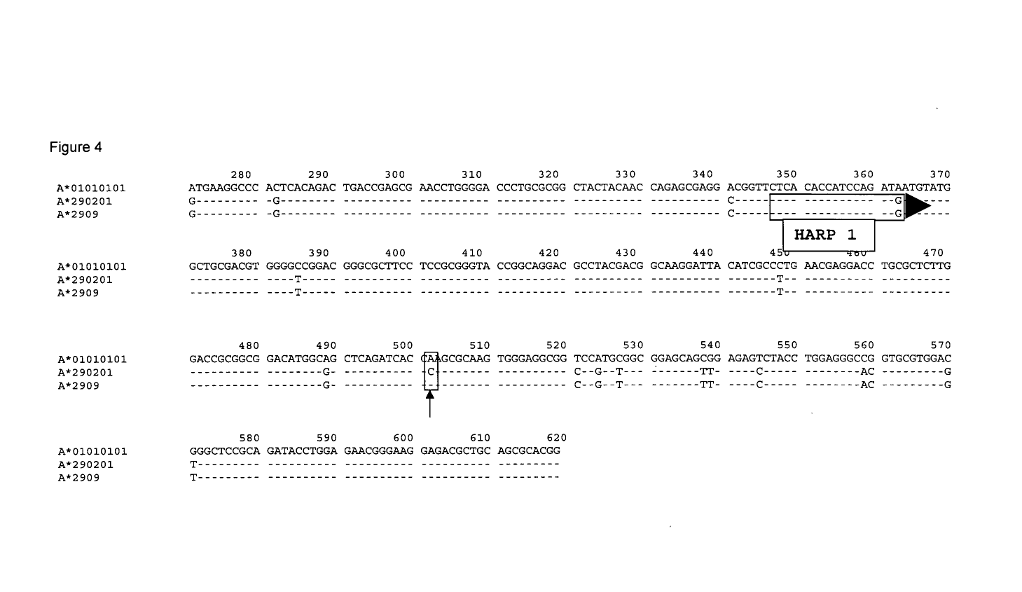

In order to obtain the precise HLA type we used a primer (HARP1) which binds to two alleles which have a sequence difference at position 502. As shown in FIG. 4, by using the information about which alleles HARP1 will bind and sequence in combination with the sequence at position 502, the precise HLA type can be determined.

High molecular weight DNA from both chromosomes of a subject was extracted from a blood sample according to the manufacturer's instructions (QIAgen). 2 microlitres of the DNA without dilution was used in a polymerase chain reaction with primers, AampF and AampR (shown in Table 1), known to simultaneously amplify DNA from both chromosomes. The upstream oligonucleotide primer is located in the 5′ untranslated region and the downstream primer is located in exon 4.

TABLE 1

| Primer | | |
|---|---|---|
| Name | Primer Sequence | Primer Function |
| AampF | TCTCCCCAGACGCCGAGGATGGCC | PCR 5′UTR |
| AampR | TGTCCTGGGTCTGGTCCTCCCCAT | PCR Exon 4 |
| X2F | TCGGACCCGGAGACTGTG | Exon 2 |
| | | (sequencing |
| | | forward) |
| X2R | GTTTCATTTTCAGTTTAGGCCA | Exon 2 |
| | | (sequencing |
| | | reverse) |
| X3F | CCTCTGYGGGGAGAAGCAA | Exon 3 |
| | | (sequencing |
| | | forward) |
| X3R | TGTTGGTCCCAATTGTCTCCCCTC | Exon 3 |
| | | (sequencing |
| | | reverse) |
| HARP1 | CTCACACCATCCAGATA | |

1 microlitre of each of AampF and AampR at a concentration of 1 picomoles per microliter was used in a PCR that also included 0.4 microlitres of polymerase enzyme (Taq Platinum Taq polymerase; Geneworks) and 17.6 microlitres of PCR buffer. The final concentrations of the constituents of the PCR buffer were 2.5 mM MgCl2, 0.2% DMSO, 67 mM Trizma Base, 16.6 mM Ammonium Sulphate, 25 mM of each dNTP

The PCR was performed in a thermal cycler (GeneAmp PCR 9700, Applied Biosystems) according to the following conditions: DNA denaturation at 96 degrees for 6 minutes followed by 35 cycles of denaturation at 96 degrees celsius for 30 seconds, oligonucleotide primer annealing at 70 degrees celsius for 30 seconds and DNA extension at 72 degrees celsius for 2 minutes. This was followed by a 10 minute extension step at 72 degrees.

At the end of the PCR 2 microlitres of sample was removed from the PCR tube and electrophoresed in an agarose gel in the presence of ethidium bromide to confirm the presence of amplified DNA of the expected size.

The remaining PCR product was purified using ExoSapIT according to the manufacturer's instructions to remove unused PCR amplification oligonucleotide primer and small non-specific products that may interfere with sequencing.

The PCR product was then sequenced using a standard cycle sequencing dye labelled di-deoxynucleotide sequencing reaction with Big Dye Terminators (BDT) v3.1 reagents (Applied Biosystems). 2 microlitres of PCR product was sequenced using 2 microlitres of each sequencing primer, X2F, X2R, X3F, and/or X3R (shown in Table 1) at 1 picomole per microlitre in a sequencing reaction which also included 8 microlitres of water, 1 microlitre of BDT reaction mix, and 7 microlitres of sequencing buffer (Applied Biosystems).

The PCR product(s) were sequenced so that exons 2 and 3 of both HLA-A alleles were sequenced simultaneously and in the same container. Alternatively, a single primer, HARP1, may be used to enable sequencing of the HLA-A allele from just one of the chromosomes.

The sequencing reactions were performed in the same thermal cycler described above under the following conditions: 25 cycles of denaturation at 96 degrees celsius for 10 seconds, oligonucleotide sequencing primer annealing at 50 degrees celsius for 5 seconds and fragment extension/termination at 60 degrees celsius for 4 minutes.

Following sequencing the DNA fragments were purified to remove unused primers and excess dye labelled di-doxynucleotides using CleanSeq. The DNA fragments were then fractionated in an Applied Biosystems 3730 XL automated capillary sequencer.

The fractionated fragments were analysed using Assign-SBT™ v 3.5 (Conexio Genomics). This software enables the simultaneous analysis of sequence obtained from alleles on each chromosome.

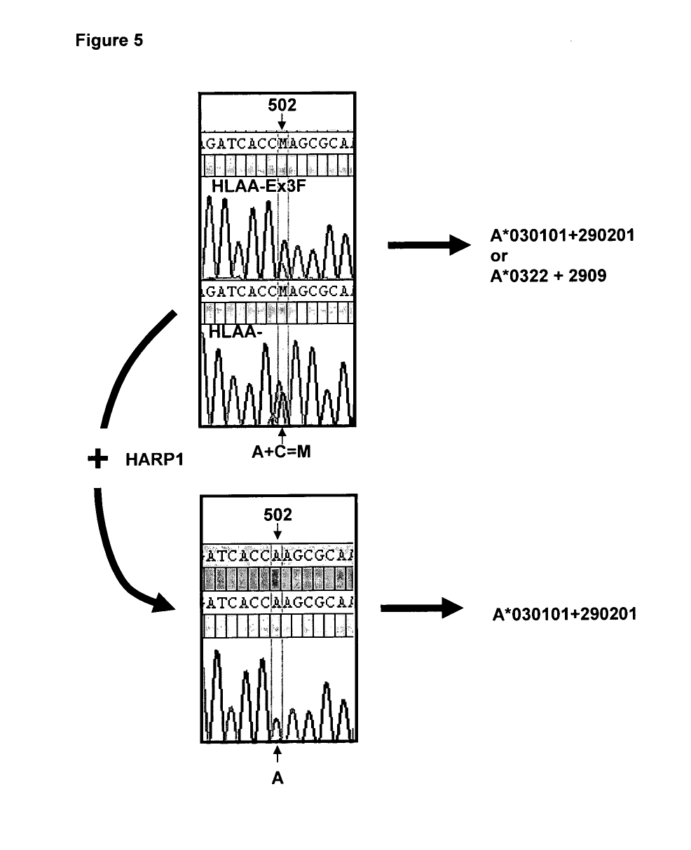

As shown in FIG. 5, the sample contained the sequence that was identical to the alleles HLA-A*03010101+290201 and A*0322+2909. The HARP1 primer is complimentary to A*290201 and A*2909 and sequences only the DNA from the chromosome that contains either A*290201 or A*2909. Identifying the “A” nucleotide at position 502 indicates that one chromosome has A*290201. Therefore the correct HLA type is A*030101+290201. If sequencing had identified a “C” at position 502 then the allele sequenced would have been A*2909 and therefore the correct type would have been A*0322+2909.

1. A method of designing an oligonucleotide for use in identifying a nucleic acid molecule, comprising the steps of:
a) identifying first and second variable regions in each of the at least two nucleic acid molecules, wherein the first and/or second variable region of each nucleic acid molecule is descriptive of that nucleic acid molecule; and
b) designing an oligonucleotide which binds to the first variable region of one nucleic acid molecule and generates information of the second variable region of that nucleic acid molecule.

2. The method of claim 1, wherein the method further comprises the step of aligning the sequence of the at least two nucleic acid molecules before step a).

3. A method of identifying a nucleic acid molecule and determining the cis/trans relationship between sequences at 2 or more sequence variable regions comprising the steps of:
a) combining an oligonucleotide which binds to a first variable region of the allele and generates information of a second variable region of the nucleic acid molecule, wherein the first and/or second variable region of the nucleic acid molecule is descriptive of that nucleic acid molecule;
b) generating information about the nucleic acid molecule; and
c) analysing the generated information to identify the nucleic acid molecule.

4. A method of identifying a nucleic acid molecule and determining the cis/trans relationship between sequences at 2 or more sequence variable regions comprising the steps of:
a) combining the information from two or more sequenced positions without including the oligonucleotide information;
b) generating information about the nucleic acid molecule; and
c) analysing the generated information to identify the nucleic acid molecule.

5. A method of HLA typing a subject, comprising the steps of:
a) combining a sample from the subject and an oligonucleotide which binds to a first variable region of an HLA allele of the subject and generates information of a second variable region of the allele, wherein the first and/or second variable region of the allele is descriptive of the allele;
b) generating the information about the allele; and
c) analysing the generated information to identify the allele, wherein identification of the allele provides the HLA type of the subject.

6. A method of treating a disease or disorder of a subject, comprising the steps of:
a) combining a sample from the subject and an oligonucleotide which binds to a first variable region of a nucleic acid molecule and generates information of a second variable region of the nucleic acid molecule, wherein the first and/or second variable region of the nucleic acid molecule is descriptive of the nucleic acid molecule;
b) generating the information about the nucleic acid molecule; and
c) analysing the generated information to identify the nucleic acid molecule, wherein identifying the nucleic acid molecule indicates how to treat the disease or disorder.

7. A method of diagnosis of a disease or disorder of a subject, comprising the steps of:
a) combining a sample from the subject and an oligonucleotide which binds to a first variable region of a nucleic acid molecule and generates information of a second variable region of the allele, wherein the first and/or second variable region of the nucleic acid molecule is descriptive of the nucleic acid molecule;
b) generating the information about the nucleic acid molecule; and
c) analysing the generated information to identify the nucleic acid molecule, wherein identifying the nucleic acid molecule provides a diagnosis of the disease or disorder.

8. The method of claim 1, wherein neither the first or second variable region is unique to the nucleic acid molecule but the combination of the first and second variable regions is unique to the nucleic acid molecule.

9. The method of claim 1, wherein the nucleic acid molecule is an allele of a gene.

10. The method of claim 2, wherein the analysis is performed by a computer program.

11. The method of claim 10, wherein the computer program is Assign-SBT™.

12. The method of claim 1, wherein the first and second variable regions are separated by 1 to 1,500 nucleotides.

13. The method of claim 1, wherein the first and second variable regions are separated by 30 to 1000 nucleotides.

14. The method of claim 1, wherein the first and second variable regions are separated by 100 to 500 nucleotides.

15. The method of claim 1, wherein the nucleic acid molecule or allele is amplified prior to step a).

16. The method of claim 4, wherein the nucleic acid molecule is an HLA gene.

17. The method of claim 4, wherein the nucleic acid molecule is HLA-DRB1.

18. The method of claim 3, wherein one oligonucleotide is used.

19. The method of claim 3, wherein at least four oligonucleotides are used.

20. The method of claim 1, wherein the oligonucleotide consists essentially of sequence CTCACACCATCCAGATA (SEQ ID NO:7).

21. The method of claim 3, wherein steps a) and b) occur in the same container.

22. An oligonucleotide having the sequence CTCACACCATCCAGATA (SEQ ID NO:7) for use in the method of claim 3.
